(19) United States
(12) Patent Application Publication
(10) Pub. No.: US 2026/0365716 A1
(43) Pub. Date: Nov. 19, 2026

(54) USE OF GELATIN IN A SUPPORT MATERIAL FOR EMBEDDED PRINTING

(71) Applicants: ROUSSELOT B.V., GENT (BE); UMC UTRECHT HOLDING B.V., UTRECHT (NL)

(72) Inventors: Joseph Hubertus OLIJVE, GENT (BE); Jan-Philip ZEGWAART, GENT (BE); Riccardo LEVATO, UTRECHT (NL); Davide RIBEZZI, UTRECHT (NL)

(21) Appl. No.: 19/477,174

(22) PCT Filed: Apr. 19, 2024
(86) PCT No.: PCT/EP2024/060818

§ 371 Date: Oct. 21, 2025

(30) Foreign Application Priority Data
Apr. 21, 2023 (BE) 2023/5306

(51) Int. Cl. C07K 14/78; B33Y 10/00; B33Y 70/00; C08J 3/24

(52) U.S. Cl. CPC C07K 14/78; B33Y 10/00; B33Y 70/00; C08J 3/24

(57) ABSTRACT

The current invention pertains to a use of gelatin in a support material for embedded printing. The current invention furthermore pertains to a method for preparing a gelatin support material for embedded printing and a gelatin support material, optionally obtainable by the method. The invention furthermore pertains to a method for embedded printing using a gelatin support material. In the context of the invention, the gelatin has an average molecular weight of 50-250 kDa and preferably is in a fluid bulk, single-phase and non-crosslinked form.

### TECHNICAL FIELD

The current invention relates to support materials for additive manufacturing, more specifically embedded printing.

### BACKGROUND OF THE INVENTION

Additive manufacturing (“3D printing”) can be used to fabricated three dimensional constructs, for instance for use as in vitro system to develop and test new therapeutic drugs, to expand and mature cells or organoids or to build engineered tissues for purpose of regenerative medicine.

Applying additive manufacturing techniques is known for printing of metals, natural or synthetic polymers, and ceramics for the manufacturing of complex constructs. Using additive manufacturing for soft biological structures has been challenging. The major challenges are the deposition of soft materials with low elastic moduli (e.g. hydrogels), which may or may not comprise cells or further biological components. The typical process of fabricating a 3D hydrogel construct involves loading a hydrogel precursor into any type of extruder system (e.g. a syringe pump extruder) and depositing it in a layer-by-layer manner to build the 3D object. New approaches involve the use of light patterns projected onto photocrosslinkable materials to create 3D objects in a layer-by-layer manner or at once in a volumetric, layerless manner.

A major obstacle to successful printing of soft and liquid print materials is the distortion of the print material due to gravity and subsequent loss of print quality. Soft and liquid print materials may encompass “bioinks”, a term used to denote specialized print material that promote formation and/or mimic the properties of living tissues or organs. Bioinks may typically consist of a mixture of living cells and a biocompatible material (e.g. a hydrogel precursor) that provides the structural support necessary for the printed structure to maintain its shape. In the absence of physical support, soft and liquid print materials including bioinks are typically challenging to print in a layer-by-layer manner and do not cure quickly enough to allow for structure stability, leading to failure of the printing process.

Embedded printing—also referred to as “Embedded Fusion Modeling (EFM)”—has been developed with the aim of solving the problem of distortion of print materials by embedding the print material inside a temporary support bath. The support bath is typically a non-Newtonian fluid that acts as a temporary physical support for the embedded print material. After printing, the deposit can be separated from the support as a fully assembled construct. The term “Freeform Reversible Embedding of Suspended Hydrogels (FRESH)” has been used to refer to a subset of embedded printing, in which the support bath is removed post printing.

A main challenge for embedded printing approaches is to provide a support material that can directly maintain the position of deposited print material structures as they are extruded and cured while still allowing for the movement of the extruder needle through the support bath during printing. To achieve this, it is thought that the support material ideally possess a yield-stress behavior, such as a Herschel-Bulkley fluid, or more specifically as a Bingham plastic, where it acts as a solid until a sufficient shear stress (the yield stress) is applied, at which point it transitions from a solid to a liquid-like behavior.

Known support materials for embedded printing include materials based on gelatin particles, i.e. in the form of “gelatin slurry”. For example, Hinton et al. (Sci Adv. 2015 Oct. 23; 1(9):e1500758) describes the following method for preparing a gelatin slurry support material:
mixing gelatin and CaCl₂ into a solution and gelling this mixture for 12 hours at 4 °C;
adding CaCl₂ at 4 °C and blending the contents to obtain a gelatin slurry;
centrifuging to remove the soluble gelatin and obtaining a slurry based on only gelatin particulates.

In the above method, the blend time can be varied to obtain gelatin microparticles in the range of typically 50-150 μm. After printing, the support material is removed by liquefying (melting) the gelatin particles, thereby releasing the printed construct.

Another method of preparing a gelatin slurry material for imbedded printing uses a coacervation approach (Lee et al., Science 365, 482-487 (2019). Similar to the mechanical blending method, the coacervation approach yields a multi-phase support bath material. Compared to the blending approach, the coacervation approach may yield relatively smaller particles (~25 μm) and therefore a higher resolution, but may generate only a relatively small quantity of particles.

Use of a gelatin slurry as support bath in embedded printing carries several limitations. A first limitation is that microparticles should first be prepared out of gelatin, meaning that the method is not readily accessible or easy to use, extensive preparation time and effort is needed, and requires very particular skill which can be associated with poor reproducibility and reliability. A second limitation is that the procedure is sensitive to variations (e.g. in the obtained microparticle distribution) between labs or gelatin batches.

As an alternative to gelatin slurry, carbopol is a commonly used polymer in support bath for embedded printing, due to its high viscosity and shear-thinning behavior. However, there are several potential disadvantages associated with using carbopol and the like in bioprinting. For instance, carbopol has been shown to have cytotoxic effects on cells, which can affect the viability and functionality of the printed tissue. Carbopol is also not isotonic, which hampers its in vivo (clinical) use. Unfortunately, carbopol is highly adhesive, which can make it difficult to separate from the printed construct and can interfere with biological responses and downstream assays and analysis. Based on the previous, there is a general preference for natural and/or more biocompatible polymers as compared to carbopol and the like.

It is an objective of the current invention to overcome one or more of the above limitations in the preparation and/or use of support materials for embedded printing.

### SUMMARY OF THE INVENTION

The object of the current invention is achieved by providing an aqueous gelatin formulation as support material, wherein the gelatin has an average molecular weight of 50-250 kDa, more preferably at least 50 kDa and below 150 kDa, most preferably 80-120 kDa. As part of the invention, the gelatin is preferably provided in a non-covalently crosslinked form and/or in a fluid bulk form in the support material. This achieves the desired yield-stress behaviour, more specifically allowing the support material to behave as a Bingham plastic, which is advantageous for embedded printing. As part of the invention, the support material is preferably provided in a single-phase form, more specifically in absence of (micro)particulate or colloidal form, such that a homogeneous material is achieved in which the composition and properties are the same throughout the entire material and wherein the gelatin (macromolecules) can still flow and behave as a liquid. As part of the invention, the support material is preferably obtained by dissolving the gelatin in an aqueous medium, preferably a salt buffer, more preferably 0.1-10× phosphate-buffered saline, and allowing (physical) crosslinking under appropriate time and temperature.

In an aspect, the current invention pertains to a method for embedding a print material in a support material, the method comprising the step of depositing the print material into the support material,
wherein the support material is an aqueous gelatin formulation,
wherein the gelatin in the aqueous gelatin formulation has an average molecular weight of 50-250 kDa.

In an aspect, the current invention pertains to a use of gelatin in a support material for embedded printing,
wherein the gelatin has an average molecular weight of 50-250 kDa.

In an aspect, the current invention pertains to a method for preparing a support material for embedded printing, comprising dissolving gelatin to a concentration of 1-20 wt. % in an aqueous medium to obtain an aqueous gelatin formulation, wherein the gelatin concentration is calculated on the total weight of the aqueous medium, and subjecting the gelatin in the formulation to crosslinking, preferably non-covalent crosslinking, wherein the gelatin in the formulation has an average molecular weight of 50-250 kDa.

In an aspect, the current invention pertains to a support material for embedded printing, obtainable by the method disclosed herein, preferably obtainable by the method comprising dissolving gelatin to a concentration of 2-10 wt. % in an aqueous medium to obtain an aqueous gelatin formulation, wherein the gelatin concentration is calculated on the total weight of the aqueous medium, and subjecting the gelatin in the formulation to non-covalent crosslinking,
wherein the gelatin in the formulation has an average molecular weight of 80-120 kDa,
wherein the non-covalent crosslinking is by subjecting the gelatin in the formulation to a temperature of 12-30 °C for 6-18 hours.

In an aspect, the current invention pertains to a support material for embedded printing,
wherein the support material is an aqueous gelatin formulation,
wherein the aqueous gelatin formulation comprises 1-20 wt. % gelatin calculated on the total weight of the aqueous gelatin formulation,
wherein the gelatin in the aqueous gelatin formulation has an average molecular weight of 50-250 kDa.

In the art, the gelatin in fluid bulk form is typically removed from the support material to obtain only gelatin micronized particles (“gelatin slurry”). Alternatively, in the art gelatin is often covalently crosslinked by functionalized gelatin and/or further crosslinking initiators in the support material.

The present inventors surprisingly show that the gelatin support material based on gelatin with certain properties possess a fluid (yield-stress) behavior providing an efficient temporary physical support for the embedded print material, even in absence of covalent crosslinking of the gelatin and even when using the gelatin in (single-phase) fluid bulk form. The present findings are unexpected, because it is taught in the art that physically crosslinked gelatin in absence of gelatin particulates is not a Bingham plastic and cannot work as a support material for embedded printing, as described in WO20150174221.

The gelatin support material of the invention may behave as a Bingham plastic and has a surprisingly high “self-healing” capacity, referring to the ability of a material to automatically repair itself after deformation during the extrusion process. The gelatin support material may act like a fluid when a print needle is moved through the support material such that the needle movement does not disturb the deposited print material.

The present inventors found that the use of soluble gelatin in the support bath increases the efficiency of the embedded printing process. No extensive preparation time and effort is needed to prepare the support material, normally associated with fabricating gelatin particles. The gelatin support bath of the invention can be obtained by simply dissolving the gelatin in an aqueous medium such as a salt buffer and allowing gelation within a certain time-frame and a certain temperature depending on the exact application. The simple and efficient procedure improves the reliability and reproducibility of the support material and the printing process.

Since no covalent crosslinking of the support material is required (it is optional as a step after printing), the user can choose to provide the support material as either a permanent or a temporary support material. The use of the gelatin support material as permanent material—e.g. as structural and/or biological component in an implantable scaffold—can be in part attributed to the excellent biocompatibility of the gelatin of the invention. In the case of a permanent support, the gelatin support material can be supplemented with an additional crosslinking initiator to allow further (covalent) crosslinking of the gelatin after embedding the print material.

The gelatin support material of the present invention can be easily removed such as by simple washing step, for instance by simply increasing the temperature past the melting point of gelatin, and without adhering to the printed object. Alternatively, the present invention offers the advantage that the support material can optionally be left in place as a permanent component of the scaffold. To this end, the gelatin is preferably functionalized with one or more chemical moieties (e.g. most preferably methacryloyl) and/or the gelatin support is supplemented with a further photo-initiator to further promote crosslinking of the hydrogel.

It is shown that the hydrogels formed from the gelatin of the invention supports can have unique mechanical properties resembling the properties of soft tissues such as the pancreas (displaying a lower stiffness with little to no stress relaxation and low storage modulus). This is an important benefit, since it has been extremely challenging to achieve 3D hydrogel mimicking the native extracellular milieu of soft tissues, in particular having low stiffness (e.g. ~10 kPa or even less) and low elasticity (e.g. elasticity of ~50% or lower) thereof, yet having the stability over a prolonged time frame as needed for developing cellularized and vascularized constructs. The hydrogels of the invention may even display inherent vasculogenic properties, which is related at least in part to the specific mechanical properties of the hydrogel. Vascularization is a primary challenge in tissue engineering, and opportunities to make (modified) gelatin hydrogels supporting vascularization are therefore of relevance. However, to date, attempts to modify the gelatin properties and functionalization to promote cellular responses and vascularization have come with the cost of greatly accelerating hydrogel degradation and showing insufficient shape stability as needed for various in vitro or in vivo biotechnological or biomedical applications.

As support material, it is found to be particularly advantageous to provide a GelMA solution with GelMA having molecular weight of 50-250 kDa, preferably 70-160 kDa, more preferably 80-120 kDa in particular in combination with a degree of functionalization (DOF) of 10-100%, preferably ~40-80%, in order to obtain a hydrogel with desirable matrix stiffness, viscoelasticity and/or stability to promote the formation of vascular connection by cultured endothelial cells.

### DETAILED DESCRIPTION OF THE INVENTION

The present disclosure pertains to a gelatin suitable as support material for embedded printing.

### (Functionalised) Gelatin

The term “gelatin” in the context of the current invention means a mixture of water-soluble proteins derived from partial hydrolysis of collagen. The gelatin of the invention may be type A gelatin, type B gelatin or a combination thereof. The hydrolysis may be performed by any acid or alkali condition, or by enzymatic hydrolysis, as known in the art. Depending on the physical and chemical methods of the partial hydrolysis, the molecular weight of the peptides can fall within a broad range (e.g. 10-95 kDa). The partial hydrolysis provides the gelatin the ability to hold water and its gelling capacity, typically distinguishing it from “collagen peptide” or “hydrolysed collagen” i.e. products obtained by further hydrolysis of collagen. The term “collagen” in the context of the current invention means an amino acid sequence comprising a repeating (Gly-X-Y) sequence, preferably comprising at least 2, 3, 4, 5, 10, 20, 50, 100, 200, 300, or 400 sequences containing the sequence Gly-X-Y, where X and Y are an amino acid residue independently chosen from each other, but X and/or Y are more preferably proline. The “collagen” preferably has a sequence found in native collagen in one or more animal species. In addition, or alternatively, the “collagen” can mean a full-length sequence or fragment or subunit thereof of (native) collagen, preferably one or more of collagen types I-XXVII, more preferably one or even more of type I, II, III, IV, V, or X collagen, even more preferably one or more of type I, II, III or IV collagen, or any combination thereof. For example, the term “collagen” may refer to an alpha-1(I), alpha-2(I), alpha-1(II) or alpha-1(III) chain, or a fragment thereof. The term “collagen” encompasses the triple helix structure as formed by three subunits as present in native collagen. The gelatin in the context of the current invention may be derived from partial hydrolysis of one or more types of collagen selected from collagen types I-XXVII, preferably one or more of type I, II, III or IV collagen, or any combination thereof. The gelatin in the context of the current invention may be derived from partial hydrolysis of one or more types of collagen of one more animals or species of animals, such as collagen of bovine (species), pig (species), chicken and fish (species), or combinations thereof.

The gelatin in the context of the current invention may include gelatin produced recombinantly or by chemical synthesis. Recombinant synthesis encompasses that a protein or peptides is encoded by recombinant DNA that is expressed in an expression system. The expression system for the recombinant peptide can be cells such as a bacterial cell (e.g., Escherichia coli, Bacillus subtilis species), yeast cell [e.g. Saccharomyces cerevisiae, Pichia pastoris or Ogataea angusta (Hansenula polymorpha), Candida bodini], fungal cell (e.g. Aspergillus oryzae, Aspergillus niger, Trichoderma reesei), mammalian cell (e.g. a CHO cell, a HeLa cell, a HEK293 cell, NS0, Sp2/0), insect cell and plant cell (e.g. tobacco, cereal, legume, fruit, vegetable).

The “gelatin” in the context of the current invention encompasses a gelatin “derivative”, including modified gelatin, preferably chemically modified gelatin. The term “modified gelatin” (also referred to as “functionalized gelatin”) in the context of the current invention encompasses gelatin modified with a chemical group or moiety (e.g. a methacryloyl group or any other chemical moiety as disclosed herein in the context of functionalised gelatins) attached to at least one amine group, at least one hydroxyl group, at least one carboxyl group and/or at least one phenol group of the gelatin. The “gelatin” as disclosed herein may be a combination (e.g. mixture) of different gelatins. For example the “gelatin” may comprise gelatins from various sources and/or of different average molecular weights. In addition or alternatively, the “gelatin” may comprise combinations of modified and/or non-modified gelatins as disclosed herein. To illustrate, the “gelatin” may comprise a non-modified gelatin together with a modified gelatin such as GelMA or GelDAT.

Typical functionalized gelatines known in the art and suitable for the present invention include for instance tyramine-functionalised gelatins as described by Wang et al. (Biomaterials. 2010. 31 (6): 1148-57), or van Hoorick et al. (Acta Biomater. 2019 Oct. 1; 97:46-73), gelatin modified using a carbodiimide mediated coupling of tyramine to gelatin (for example described in WO2006/010066) or desaminotyrosine (sometimes abbreviated as DAT) functionalized gelatin (e.g. as described in WO2022258763A1). The compound desaminotyrosine is also known as 3-(4-hydroxyphenyl)propanoic acid.

In a preferred embodiment, the gelatin is functionalised gelatin. More preferably, the functionalised gelatin is selected from the group consisting of gelatin-methacrylamide, gelatin acryloyl, gelatin desaminotyrosine, gelatin-methacryloyl, gelatin-aminoethyl, gelatin-methacryloyl-aminoethyl, gelatin-styrene, gelatin tyramine, gelatin-methacrylamidedopamine, gelatinmethacryloyl-acyl, gelatin-acrylamide, gelatin-3,30,4,40-benzophenone tetra carboxylic dihydroxyethylmethacrylate, gelatin with boc-protected primary amines and aminoethylmethacrylate coupled to the carboxylic acids, methacrylated poly(ethylene glycol)-modified gelatin, aminated gelatin, aminated-thiolated-gelatin, gelatin modified with cysteine and 2-mercaptopyrimidine-4,6 diol, gelatin-cys, gelatin-poly(ethylene glycol) cysteine, gelatinthiobutylamidine modified with 2-mercaptonicotinic acid, thiolated gelatin, gelatin-thiobutyrolacton, gelatin-tyramine, gelatin/tyramine/heparin, gelatin furfuryl amine, gelatin furfuryl isocyanate, gelatin furfuryl glycidyl ether, gelatin-5-[2-(5-methyl furylene vinylene)]furancarboxyaldehyde, gelatin-anthracene, gelatin-nitrocinnamate, gel-furan, gelatin furfuryl glycidyl ether, gelatin-norbornene, gelatin tetrazine, gelatin-pentenoate, gelatin allylglycidyl ether, and gelatin-vinylester, or any combination thereof.

In a preferred embodiment, the gelatin is gelatin methacryloyl (i.e. GelMA) or gelatin desaminotyrosine (i.e. GelDAT), or a combination thereof.

In the context of the current invention, the term “gelatin methacryloyl” (i.e. GelMA) means gelatin having free amines and/or free hydroxyls that have been substituted with at least one methacrylamide group and/or at least one methacrylate group. The term “gelatin methacryloyl” can be used interchangeably with the terms “gelatin modified with a methacryloyl group”, “methacryloyl-modified gelatin”, “methacryloyl-substituted gelatin” or “methacryloyl-gelatin” or “gelatin methacryloyl”, by which it is also referred to. Gelatin may comprise amino acid side chains that terminate in amines (e.g., lysine, arginine, asparagine, glutamine) or hydroxyls (e.g., serine, threonine, aspartic acid, glutamic acid, tyrosine, hydroxyproline), and in the context of the current invention one or more of these terminal amines and/or hydroxyls can be substituted with methacryloyl groups to produce GelMA comprising methacrylamide and/or methacrylate groups, respectively. The GelMA is preferably obtained by reacting gelatin with methacrylic anhydride.

In the context of the current invention, the term “gelatin desaminotyrosine” (i.e. GelDAT) means gelatin functionalised via an amide bond with 3-(4-hydroxyphenyl)-propionic acid.

In an embodiment, the gelatin is functionalised via an amide bond with a carboxylic acid moiety R—(CH₂)ₙ—COOH wherein n is an integer form 1 to 10, wherein R is selected from the group consisting of 4-phenol, norbornenyl or SH, more preferably wherein the carboxylic acid moiety is one or more of 3-(4-hydroxyphenyl)-propionic acid, 3-(SH)-propionic acid, and 2-(5-norbornenyl)-acetic acid, most preferably 3-(4-hydroxyphenyl)-propionic acid.

The gelatin of the invention may be (further) modified and/or functionalized, e.g. with an acetyl group or moiety, a phenol group or moiety, a thiol group or moiety, a norbornene group or moiety, a tetrazine group or moiety, an azide group or moiety, a furan group or moiety, a galactosyl group or moiety, or any combination thereof.

The term “degree of functionalization” (DOF) of gelatin refers to the percentage of functionalized primary amine groups and hydroxyl groups over total primary amine groups and hydroxyl groups. The term “functionalised group” refers to a group modified with a one or more chemical moieties disclosed herein in the context of the functionalised gelatins. As used herein, the “degree of methacrylamide substitution” refers to the percentage of free amine groups in the gelatin that have been substituted with a methacrylamide group. As used herein, the “degree of methacrylate substitution” refers to the percentage of free hydroxyl groups in the gelatin that have been substituted with a methacrylate group. As used herein, the “degree of carboxylic acid substitution” refers to the percentage of free amine groups in the gelatin that have been substituted with a carboxylic acid group. As used herein, the “degree of desaminotyrosine substitution” refers to the percentage of free amine groups in the gelatin that have been substituted with a desaminotyrosine group.

The degree of functionalization of gelatin can be determined by methods known per se. For example, the Fe(III)-acetohydroxamic acid method can be used for determining methacrylation at hydroxyl groups. The Flabeeb method (trinitrobenzenesulfonic acid (TNBS)-based spectrophotometric determination of (meth)acrylamide; Flabeeb 1996. Anal. Biochem. 14:328-336), 1 FI-NMR and the fluoraldehyde assay (also known as o-Phthaldialdehyde (OPA)-based fluorometric determination of methacrylamide) can be used for determining (meth)acrylation at amine groups. One can also use a combination of the aforementioned methods, such as a combination of a fluoraldehyde assay for quantifying amine group conversion and a Fe(III)-acetohydroxamic acid method for quantification of methacrylate groups, which allows to determine the ratio of methacrylate to methacrylamide groups. In embodiments, the fluoraldehyde assay is used for determining the degree of methacrylamide substitution. The Habeeb method, 1H-NMR and the fluoraldehyde assay can be used for determining carboxylic acid substitution at amine groups. One can also use a combination of the aforementioned methods, such as a combination of a fluoraldehyde assay for quantifying amine group conversion and a Fe(III)-acetohydroxamic acid method for quantification of hydroxyl groups. In embodiments, the fluoraldehyde assay is used for determining the degree of carboxylic acid substitution by determination of the free amines.

The DOF values disclosed herein can be used also to indicate the degree of methacrylamide substitution, degree of methacrylate substitution, degree of carboxylic acid substitution or degree of desaminotyrosine substitution.

In embodiments, the gelatin has a DOF of at least 10%, 20%, 30%, 40%, 50% or 60% and/or less than 100%, 90%, 80%, 70%, 60%, 50% or 40%. In a preferred embodiment, the gelatin has a DOF 40-80%, preferably 50-70%.

The present inventors found that the self-healing capacity of a gelatin support material is dependent on the gelatin molecular weight.

In embodiments, the gelatin has a molecular weight of at least 40 kDa, 50 kDa, 60 kDa, 70 kDa, 80 kDa, 90 kDa, 95 kDa, 100 kDa, 101 kDa, 102 kDa, 103 kDa, 104 kDa, 105 kDa, 106 kDa, 107 kDa, 108 kDa, 109 kDa, 110 kDa, 120 kDa, 130 kDa, 140 kDa, 150 kDa or 160 kDa and/or less than 240 kDa, 230 kDa, 220 kDa, 210 kDa, 200 kDa, 190 kDa, 180 kDa, 170 kDa, 160 kDa, 150 kDa, 140 kDa, 130 kDa, 120 kDa, 110 kDa, 109 kDa, 108 kDa, 107 kDa, 106 kDa, 105 kDa, 104 kDa, 103 kDa, 102 kDa, 101 kDa, 100 kDa, 90 kDa, 80 kDa or 70 kDa.

In a preferred embodiment, the gelatin has an average molecular weight of 50-250 kDa. In a preferred embodiment, the gelatin has an average molecular weight of 70-160 kDa. In a preferred embodiment, the gelatin has an average molecular weight of least 50 kDa and below 150 kDa. In a preferred embodiment, the gelatin has an average molecular weight of least 70-130 kDa. In a preferred embodiment, the gelatin has an average molecular weight of 80-120 kDa.

Gelatin does not constitute a uniform protein molecule, but comprises a variable amount of protein molecules of variable length. The molecular weight (i.e. MW) in the context of the current invention is preferably the weight-average molecular weight.

The molecular weight of gelatin can be measured by size exclusion high performance liquid chromatography (HPLC) techniques known in the art (e.g. see Olijve et. al. 2000. Journal of Colloid and Interface Science 243: 476-482). In addition or alternatively, a preferred way of measuring the (average) molecular weight of gelatin is by high performance size exclusion chromatography (HPSEC). The following protocol is a preferred HPSEC protocol: The Agilent HPLC, 1260 Infinity series (G1316A, G1329B, G1311C, G1315D) with a TSKgel SWXL precolumn und a G2000SWXL column (Tosoh Bioscience) is used. Analysis is performed with the WinGPC software (PSS). The eluent is 100 mM phosphate buffer pH 5.3. Samples are eluted from the column (e.g. 0.5 mL/min, isocractic) and monitored with UV detection (e.g. 214 nm, analysis time: 40 min per injection+180 min equilibration). Calibration is performed with the Narrow Calibration Standard (Low FILK).

It may be advantageous for the biocompatibility and/or vasculogenic properties of the hydrogel to use a gelatin with a high purity. In a preferred embodiment, the gelatin has a lipopolysaccharide content of less than 1000 EU/g, preferably less than 100 EU/g, more preferably less than 10 EU/g. Widely-used and preferred methods for measuring endotoxin (i.e. lipopolysaccharide, LPS) level (e.g. for determining EU/g or EU/ml) in the context of the current invention are the Limulus Amebocyte Lysate (LAL) test or the recombinant Factor C (rFC) test, which the skilled person is familiar with. The term EU is known in the art and reflects ‘endotoxin units’.

In a preferred embodiment, the gelatin is GelMA, wherein the GelMA comprises less than 150 ppm methacrylic acid (MAA), preferably less than 50 ppm MAA, more preferably less than 25 ppm MAA. Upon reacting gelatin with methacrylic anhydride to prepare GelMA, methacrylic anhydride (MAAH) and/or methacrylic acid (MAA) can be present in the gelMA, which are considered hazardous. MAA may cause adverse effects at the site of application, depending on the concentration and frequency or time of exposure. Unreacted methacrylic anhydride and methacrylic acid can be (partly) removed from the reaction mixture by dialysis against distilled water. The MAA content can be determined for instance on a sample of the GelMA dissolved in 50 mM phosphate buffer (pH 9.5), ultrafiltration of the dissolved sample (e.g. using 10-kDa Amicon Ultra Centrifugal Filters) and HPLC analysis of the filtrate.

### Gelatin Support Material

According to the present invention, a gelatin support material is provided.

The gelatin support material is preferably an aqueous gelatin formulation.

The gelatin support material is suitable as support material in additive manufacturing, more specifically in embedded printing.

The present inventors found that the gelatin support material of the invention provides good yield-stress behavior, as needed for embedded printing. Using the gelatin of the invention, the necessary yield-stress behavior can be achieved already even by solely physically crosslinking the gelatin. This can eliminate the need for covalently crosslinking the gelatin and/or use of further crosslinking initiators or rheology modifiers. The gelatin support material of the invention offers, among others, advantages in terms of for example ease of production, short preparation time, high reproducibility, good biocompatibility and high scalability of the method. Since the method does not rely on functionalised gelatins (which are optional in the support material according to the invention), this reduces the costs and allows larger volumes of support materials to be used. The (thermo)reversibility of the crosslinking of the gelatin is advantageous, because the support material can be easily separated from the print material (for instance by simple melting). The gelatin support of the invention is also found to have inherent vasculogenic properties which supports its use as permanent support material, i.e. forming a biological and/or structural component of the final construct.

Preferably the gelatin is provided in the support material in a fluid bulk form, wherein the non-covalent crosslinks may allow the gelatin (macromolecules) to still flow and behave as a liquid. The non-covalent crosslinks can still provide some stability to the gelatin solution, such as preventing it from completely dissolving or separating into its individual molecules.

In an embodiment, the gelatin support material is and/or or behaves as a “Herschel-Bulkley fluid”, meaning a type of non-Newtonian fluid that exhibits a yield stress, which means it will not flow until a certain amount of stress or force is applied to it. Once the yield stress is exceeded, the fluid will begin to flow, but its viscosity will not remain constant and will instead vary with the shear rate applied. In an embodiment, the gelatin support material is and/or behaves as a “Bingham plastic” or Bingham plastic-like material, meaning that it is a solid material when not perturbed, but shear thins and provides minimal resistance when a printing object (e.g. needle, extruder) moves through it. Herschel-Bulkley fluids and Bingham plastics are viscoplastic materials included in the “shear-thinning” or “yield-stress fluid” category.

In a preferred embodiment, the gelatin in the support material is non-covalently crosslinked.

In a preferred embodiment, the gelatin in the support material is physically crosslinked.

In a preferred embodiment, the gelatin in the support material is reversibly crosslinked.

In a preferred embodiment, the gelatin in the support material is thermoreversibly crosslinked.

In an embodiment, the support material is a gel. In an embodiment, the support material is a hydrogel. In an embodiment, the support material is a hydrogel precursor.

Gelatin can undergo various types of covalent and non-covalent crosslinking.

Covalent crosslinking in gelatin can occur for instance through the formation of amide bonds between amino acid residues, including lysine, hydroxylysine, and histidine, with other gelatin molecules. These crosslinks can be formed through a variety of chemical reactions, such as oxidative, thermal, or enzymatic treatments. The resulting crosslinked gelatin networks can exhibit enhanced mechanical strength, thermal stability, and resistance to enzymatic degradation. Typically, covalent bonding is (further) induced through use of crosslinking agents, which, depending on the specific crosslinking agent used, may lead to amide bonds, Schiff bases, ester bonds or disulphide bonds. In the case of the GelMA for example, Irgacure 2959 is commonly used as crosslinking agent, which generates free radicals that cause methacrylate groups on the GelMA molecules to react with each other through a radical-mediated addition reaction, forming covalent bonds between the polymer chains. Specifically, the carbon-carbon double bonds in the methacrylate groups can undergo addition reactions with each other, leading to the formation of covalent bonds and the creation of a three-dimensional network structure.

Examples of non-covalent crosslinking in gelatin are those occurring through various weak interactions, including hydrogen bonding, electrostatic interactions, ionic bonds and hydrophobic interactions. These interactions can lead to the formation of physical networks between gelatin molecules.

There are several methods known in the art which can be used to determine presence of covalent or non-covalent crosslinking in gelatin, including but not limited to Fourier Transform Infrared (FTIR) Spectroscopy, Differential Scanning Calorimetry (DSC) or Nuclear magnetic resonance (NMR) spectroscopy. FTIR can be used to determine the presence of covalent or non-covalent crosslinking in gelatin based on the vibrational spectra. For example, in case of covalent crosslinking, the presence of amide bonds, which are typically formed through the reaction of the amine groups on lysine residues with carboxylic acid groups on other gelatin molecules, can be detected through the characteristic absorption bands at around 1650 cm⁻¹ and 1540 cm⁻¹ in the FTIR spectrum. Non-covalent crosslinking in gelatin, on the other hand, can be detected through changes in the intensity and position of certain peaks in the FTIR spectrum. For example, hydrogen bonding between different gelatin molecules can lead to changes in the position and intensity of the amide I and II bands, which correspond to the stretching and bending vibrations of the peptide bonds in the protein backbone. Additionally, the presence of hydrophobic interactions can lead to changes in the intensity and position of the CH and CH₂ bands. NMR spectroscopy can be used to distinguish between covalent and non-covalent crosslinking in gelatin based on the chemical shift values and peak broadening of certain NMR signals. In gelatin, the amino acid residues lysine and hydroxyproline are often involved in crosslinking, either through covalent bonding or non-covalent interactions. The presence of covalent crosslinking can for instance be detected through the observation of new peaks in the NMR spectrum, corresponding to the chemical shift values of the crosslinking products. Non-covalent crosslinking in gelatin, on the other hand, can for instance be detected through peak broadening in the NMR spectrum. This is due to the fact that non-covalent interactions between gelatin molecules are typically weaker than covalent bonds, leading to faster exchange rates between different conformations.

In a less preferred embodiment, the (crosslinked) gelatin can be gelatin crosslinked by a method that is typical for functionalised gelatins and/or gelatin crosslinked by polymerizing reactive functionalities added to the gelatin (for instance as describe by van Hoorick et al. Acta Biomater. 2019 Oct. 1; 97:46-73). The (crosslinked) gelatin in a less preferred embodiment can be gelatin crosslinked by one or more of the following crosslinking methods: step-growth polymerization, thiol-ene photo click crosslinking, Diels-Alder based click systems, disulphide linkage-based crosslinking, crosslinking based on Schiff's-base reaction, crosslinking based on photo-oxidation based systems, crosslinking based on photo-reversible systems and enzymatic crosslinking. In addition or alternatively, in a less preferred embodiment, the (crosslinked) gelatin according to the present invention may be any crosslinking that requires additional crosslinking initiator to be added to achieve crosslinking.

The term “reversible crosslinking” in gelatin in the context of the current invention means crosslinking that can be broken or undone under conditions, including physiological conditions, such as changes in temperature, pH, or ionic strength. The crosslinks formed in reversible crosslinking are typically non-covalent, such as hydrogen bonds or ionic bonds, that can be disrupted by altering the conditions. The term “non-reversible crosslinking” in gelatin in the context of the current invention means crosslinking that cannot be broken or undone under conditions, including physiological conditions, such as changes in temperature, pH, or ionic strength. The crosslinks formed in non-reversible crosslinking are typically covalent bonds. The term “thermoreversible crosslinking” in the context of the current invention means that the crosslinks can be disrupted by increasing the temperature above a certain threshold, causing the gelatin to transition from a crosslinked, solid state to a solubilized, liquid state. When the temperature is lowered again, the crosslinks can reform, causing the gelatin to return to its crosslinked, solid state. In the context of the current invention, thermoreversibly crosslinked gelatin can for instance be discriminated from non-thermoreversible crosslinked gelatin by subjecting it to a temperature cycle. If the gelatin is thermoreversible, it will transition from a solid to a liquid state upon heating and return to a solid state upon cooling. In contrast, if the gelatin is non-thermoreversible, it will remain in a solid state upon heating and cooling, as the crosslinks cannot be disrupted and reformed by temperature changes. Other techniques, such as rheological measurements, can also be used to characterize thermoreversibly crosslinked gelatin. For example, thermoreversibly crosslinked gelatin will typically exhibit a change in viscosity or elasticity in response to temperature changes, whereas non-thermoreversibly crosslinked gelatin will not.

In a preferred embodiment, the support material and/or aqueous gelatin formulation in the context of the current invention is a single-phase material. In the context of the current invention, a “single-phase material” means a homogeneous material in which the composition and properties are the same throughout the entire material. For instance, in the case of a gelatin fluid bulk, the gelatin molecules or crosslinked network of gelatin molecules are uniformly dispersed in a liquid solvent, forming a homogeneous solution. In the context of the current invention “a multi-phase material” means a material consisting of two or more distinct phases, each with its own composition and properties. An example of a multi-phase material in the context of the current invention is a colloidal suspension of gelatin particles obtained by crosslinking gelatin and comminuted to form (micro)particles, which are suspended in an aqueous solution to form a colloidal suspension. The (micro)particles are distinct phases within the suspension, with their own composition and properties, and the aqueous solution is the continuous phase.

In an embodiment, the support material and/or aqueous gelatin formulation is free of microparticles. As used herein, the term “microparticle” means a particle with an (average) diameter of 1-100 micrometer. In an embodiment, the amount of microparticles in the support material and/or aqueous gelatin formulation is less than by 10% by weight, preferably less than 1% by weight, more preferably less than 0.1% by weight, most preferably less than 0.01% by weight, calculated on the support material and/or aqueous gelatin formulation. A suitable method to determine the presence of a microparticles in a suspension is centrifugating the suspension to cause microparticles to settle out of suspension and subsequently determining the particle size, for instance as described by Hinton et al. (Sci Adv. 2015 Oct. 23; 1(9):e1500758).

In a preferred embodiment, the support material and/or aqueous gelatin formulation in the context of the current invention is a fluid bulk material and/or fluid bulk gelatin. The term “fluid bulk” in the context of the current invention, means a material which can flow and behave as a liquid, and has no distinct three-dimensional network structure. Non-covalent crosslinking of gelatin can for instance lead to the formation of a fluid bulk gelatin solution. In a fluid bulk gelatin solution, the non-covalent crosslinks may allow the gelatin (macromolecules) to still flow and behave as a liquid, however the non-covalent crosslinks can still provide some stability to the gelatin solution, such as preventing it from completely dissolving or separating into its individual molecules.

In embodiments, the support material and/or aqueous gelatin formulation comprises (essentially) no further crosslinking agents for gelatin, wherein the crosslinking agent is preferably one or more selected from the group consisting of glutaraldehyde, formaldehyde, acetaldehyde, α-Ketoglutaric acid, glyoxal, transglutaminase, a cabodiimide (e.g. EDC, DCC), tannic acid and genipin. In embodiments, the support material comprises less than 1 wt. %, preferably less than 0.1 wt. %, more preferably less than 0.01 wt. %, even more preferably less than 0.001 wt. %, most preferably less than 0.0001 wt. % of the crosslinking agent for gelatin, calculated on the total weight of the support material.

In embodiments, the support material comprises 0.1-40 wt. %, preferably 1-20 wt. %, more preferably 2-10 wt. %, most preferably 3-7 wt. % gelatin as disclosed herein, calculated on the total weight of the support material. In embodiments, the support material comprises one or more salts, wherein the salt is preferably a chloride salt and/or a phosphate salt. In embodiments, the support material comprises a salt buffer. The salt buffer may be phosphate-buffered saline, such as 0.01-100×, preferably 0.1-10×, more preferably 0.5-2× phosphate-buffered saline. The support material preferably has a pH of 5-9, more preferably 6-8.

It is typically considered that the ionic strength of gelatin support baths needs to be optimized to achieve the desired rheological properties and printing performance. Ionic strength refers to the concentration of ions in a solution and is thought to significantly influence the gelation behavior, viscosity, and mechanical properties of gelatin gels used in support baths.

Ions, most typically calcium ions (Ca²⁺) are commonly added to modulate the ionic strength of the gelatin solution (e.g. as calcium-containing salt such as one or more of [CaCl₂, CaSO₄, CaCO₃, Ca(OH)₂)]. In addition to calcium ions, other divalent cations such as magnesium ions (Mg²⁺) or zinc ions (Zn²⁺) can also be used to modulate the ionic strength of gelatin solutions for 3D printing, although calcium ions are more commonly employed due to their strong crosslinking capabilities and compatibility with gelatin.

The present inventors surprisingly found that if a gelatin with appropriate molecular weight is selected, a further modulation of the rheological behaviour of the support bath may not be necessary and for instance can be used in simple PBS and with neutral osmolarity.

In an embodiment, the support material disclosed herein does not comprise added calcium ions (Ca²⁺), e.g. no added calcium-containing salt. In an embodiment, the support material disclosed herein comprises no calcium-containing salt, wherein the calcium-containing salt may be CaCl₂, CaSO₄, CaCO₃ and/or Ca(OH)₂. In an embodiment, the support material disclosed herein comprises less than 10 mM, preferably less than 5 mM, more preferably less than 1 mM calcium-containing salt, wherein the calcium-containing salt may be CaCl₂, CaSO₄, CaCO₃ and/or Ca(OH)₂.

In an embodiment, the support material disclosed herein has a total ionic strength of 0-200 mM, preferably 50-180 mM.

### Method for Embedded Printing

According to the present invention, a method of additive manufacturing is provided, using the gelatin support material disclosed herein. In a preferred embodiment, the additive manufacturing is embedded printing, more preferably extrusion-based embedded printing.

The term “additive manufacturing” in the context of the current invention encompasses “layer-by-layer additive manufacturing” and “volumetric additive manufacturing”. “Layer-by-layer additive manufacturing” means any process of creating physical objects by adding successive layers of material, such as in a digital or computer-aided design (CAD) environment. “Volumetric additive manufacturing” means a projection of a series of 2D patterned optical light fields within a volume of a photopolymer. “Additive manufacturing can herein be used interchangeably with “3D printing”. The additive manufacturing in the context of the current invention can be one or more of embedded printing, Digital Light Processing (DLP), lithographic printing, stereolithography, extrusion-based printing, inkjet printing, laser-based methods and melt electrowriting. The term “bioprinting” in the context of the current invention means the additive manufacturing with the use of living cells and/or one or more biological factors (e.g. as disclosed herein), and which for instance can form and/or aid in formation of a biological tissue after implantation into the body. The term “tissue engineering” in the context of the current invention means the manufacturing of a functional substitute for damaged, diseased, or lost tissue or organ.

The term “embedded printing” in the context of the current invention means the printing of a print material into a physical support material (also referred to as “support bath” herein). The support material reduces the gravitational force which otherwise exists when printing directly in air. The support material in embedded printing may hold up overhanging or otherwise unsupported features of the printed object. The support material is typically a non-Newtonian fluid. The support material may be temporary (also referred to as a “fugitive support” or “sacrificial support”), meaning that it is separated from the embedded print material. The support material may be permanent, meaning that it is left intact with the embedded print material (e.g. forming a biological and/or structural component in the end construct). The term “extrusion-based embedded printing” means embedded printing wherein the print material is fed through a nozzle that penetrates the support material, thereby allowing the print material to be deposited into the support material. The nozzle can be part of any injector, needle, or extruder system, typically located at the tip thereof. The “extrusion-based embedded printing” encompasses the extrusion of a print material, via any printing or extrusion system that penetrates the support material. The terms “extrusion” or “extruding” can herein be used interchangeably and mean the same as the terms “injection”, “injecting” and “printing”. The term “extruder” can herein be used interchangeably and means the same as the term “injector”.

As part of the method, an extruder can be lowered into the support material and moved around to deposit print material in arbitrary 3D geometries. Due to the support material, the deposited print material can stay in place when the tip of the extruder moves away, thus forming the 3D printed object. The support material and/or the print material may be (further) crosslinked, preferably covalently crosslinked, during and/or after depositing the print material. The support material and/or the print material may comprise one or more crosslinking initiators and/or one or more crosslinking blockers to direct the efficiency or amount of crosslinking. Optionally, the method for embedding the print material in the support material involves selecting a parameter of the material that to promote transition of the print material form the fluid phase to the solid phase, wherein the parameter is preferably one or more of composition, pH, ionic strength, buffering capacity and enzymatic activity. The parameter may be adjusted based on the type of print material. Optionally, the support material includes calcium chloride to provide divalent cations to crosslink the hydrogel as the hydrogel is extruded out of the extruder.

In an embodiment, the print material is embedded in the support material at a location where the printed material is deposited.

The support material can be either a permanent or a temporary support material. If the support material is a temporary support, it can be removed and/or separated from the print material, e.g. after the crosslinking of the print material. The separating can for instance include a washing step. In addition or alternatively, separating the support material and the print material can be achieved by adjusting the temperature of the support material (e.g. cooling and/or heating), preferably by heating (e.g. to melt the gelatin). A print structure can be printed in any direction in 3D space, including layer by layer in the XY plane, layer by layer in a non-XY plane, such as the XZ plane and/or a plane at any angle. A structure may also be printed in a non-planar fashion in a curved path.

In a preferred embodiment, the method comprises the step of depositing the print material into the support material. In an embodiment, the depositing is by injection. In an embodiment, the depositing is by extrusion.

In a preferred embodiment, the depositing involves depositing the print material layer by layer in an XY plane and/or depositing the print material layer by layer in an XZ plane.

In an embodiment, the print material is crosslinked, preferably covalently crosslinked, after depositing the print material in the support material.

In an embodiment, the support material is separated from the print material after the crosslinking, preferably the covalent crosslinking, of the print material.

In an embodiment, the gelatin in the support material is covalently crosslinked after depositing the print material.

In an embodiment, the support material and the print material are not separated after covalent crosslinking of the gelatin in the support material.

### Print Material in Embedded Printing

In a preferred embodiment, the print material comprises a hydrogel precursor.

In a preferred embodiment, the print material comprises a bioink.

The term “hydrogel” in the context of the current invention means a network of hydrophilic polymer chains forming a gel. The term “gel” denotes a substantially dilute crosslinked system, preferably covalently crosslinked system, which exhibits no flow when in the steadystate. The term “hydrogel precursor” in the context of the current invention means a polymeric solution wherein polymer is not yet crosslinked, preferably not yet covalently crosslinked, and/or not yet forming a gel. The hydrogel precursor can be converted into a hydrogel with processes well-known in the art. Typically, upon exposure to light preferably in the presence of a photoinitiator, one or more chemical moieties (e.g. methacryloyl, desaminotyrosine) on a gelatin molecule can react with one or more chemical moieties (e.g. methacryloyl, desaminotyrosine) on another gelatin molecule to crosslink the gelatin.

The term “bioink” in the context of the current invention means any print material that promotes formation of a tissue or organ and/or mimics the properties of living tissues or organs. A “bioink” typically encompasses biocompatible print materials (e.g. a hydrogel precursor) to provide structural support, and furthermore living cells and/or one or more biological factors to promote tissue formation. In a preferred embodiment, the bioink comprises a hydrogel precursor as disclosed herein, and furthermore one or more cells as disclosed herein and/or one or more biological factors as disclosed herein.

In an embodiment, the hydrogel and/or hydrogel precursor comprises a natural polymer, a synthetic polymer, or a combination thereof. Natural polymers suitable for the invention may be one or more selected from the group consisting of collagen, gelatin, alginate, fibrin, collagen, hyaluronic acid, chitosan, silk, silk fibroin and matrigel. Synthetic (co-)polymers suitable for the invention may be one or more selected from the group consisting of polyethylene glycol (PEG), poly(lactic-co-glycolic acid) (PLGA), polyurethane (PU), polycaprolactone (PCL), polyvinyl alcohol (PVA), polyethylene oxide (PEO) and polyethyleneimine (PEI), or combinations thereof.

In an embodiment, the print material comprises one or more crosslinking initiators and/or one or more crosslinking blockers. Suitable crosslinking initiators include one or more photoinitiators and/or enzymatic crosslinking initiators known in the art.

The term “photoinitiator” in the context of the current invention refers to any chemical compound, or a mixture of compounds, that decomposes into free radicals when exposed to light such as Norrish type I initiators, e.g. ultraviolet light (UV) or visible light (VIS). Non-limiting examples of ultraviolet photoinitiators in the context of the current invention include 1-[4-(2 hydroxyethoxy)-phenyl]-2-hydroxy-2-methyl-1-propane-1-one (also known under the trade name Irgacure 2959), lithium phenyl-2,4,6-tri-methylbenzoylphosphinate (LAP), Eosin Y, riboflavin/triethanolamine, vinyl caprolactam, dl 2,3-diketo-1,7,7-trimethylnorcamphane (CQ), 1-phenyl-1,2-propadione (PPD), 2,4,6 trimethylbenzoyl-diphenylphosphine oxide (TPO), bis(2,6-dichlorobenzoyl)-(4 propylphenyl)phosphine oxide (Ir819), 4,4′-bis(dimethylamino)benzophenone, 4,4′bis(diethylamino)benzophenone, 2-chlorothioxanthen-9-one, 4 (dimethylamino)benzophenone, phenanthrenequinone, ferrocene, dipheny 1(2,4,6 trimethylbenzoyl)phosphine oxide/2-hydroxy-2-methylpropiophenone (50/50 blend), dibenzosuberenone, (benzene) tricarbonylchromium, resazurin, ruthenium, resorufin, benzoyltrimethylgermane (Ivoceri®), sodium persulfate, derivatives thereof, and any combination thereof. The photoinitiator may be any Norrish Type II photoinitiator, meaning a photoinitiator which absorbs light energy and transfers it to a co-initiator molecule, which then generates free radicals and initiates the polymerization process.

The crosslinking blocker may be one or more selected from the group consisting of a photoblocker, enzymatic crosslinking blocker and a radical scavenger. The photoblocker is preferably one or more selected from the group consisting of new coccine, tartrazine, quinoline yellow, sudan II and phenothiazine, or any combination thereof.

The crosslinking initiator is preferably present in print material in an amount of 0.001-5 wt. %, preferably 0.01-1 wt. %, more preferably 0.1-0.5 wt. %, calculated on the total weight of the print material.

The composition of the print material according to the invention can vary depending on the application (e.g. type of tissue being printed) and can include extracellular matrix (ECM) components, growth factors, and various types of cells. In addition or alternatively, the print material may comprise a metal material, a ceramic material and/or a polymer material, or any combination thereof.

In embodiments, the print material comprises one or more biological factors selected from the group consisting of cells, an organoid, an extracellular vesicle, serum, a serum protein, a growth factor, an extracellular matrix component and decellularized extracellular matrix. The term “serum” as disclosed herein encompasses human and animal serum, including but not limited to fetal bovine serum (FBS), fetal calf serum (FCS), or newborn bovine serum (NBS), newborn calf serum (NBCS). The term “serum” as disclosed herein encompasses heat-inactivated serum. The term “serum protein” in the context of the current invention encompasses human serum proteins and animal serum proteins, preferably human serum albumin (HSA) or bovine serum albumin (BSA). The “growth factor” includes one or more of vascular endothelial growth factor (VEGF), basic fibroblast growth factor (bFGF), platelet-derived growth factor (PDGF) and angiopoietin-1 (ANG-1). The extracellular matrix component is preferably one or more selected from the group consisting of collagen, fibronectin, laminin, glycosaminoglycan and hyaluronic acid. The extracellular matrix components is preferably in a recombinant form. The term “decellularized extracellular matrix” means extracellular matrix subjected to one or more of physical stress and chemical/enzymatic agents to remove cellular components, leaving behind only the noncellular extracellular matrix. The cells may include one or more of vascular cells, perivascular cells, endothelial cells, stromal cells, stem cells and smooth muscle cells (SMCs), such as one or more selected from the group consisting of endothelial cells (ECs), embryonic stem cells (ESs), human umbilical vein endothelial cells (HUVECs), endothelial-colony-forming cells (ECFCs), mesenchymal stem cells (MSCs, also referred to as “multipotent stromal cells”). The cells herein are preferably human cells in the context of the current invention. The cells herein encompass cell lines. The cells herein may be derived from pluripotent stem cells, as known in the art (Jang et al. Am J Pathol. 2019 March; 189(3):502-512, Dupuis et al. World J Stem Cells. 2021 Aug. 26; 13(8): 1094-1111).

In embodiments, the print material comprises one or more salts, wherein the salt is preferably a chloride salt and/or a phosphate salt. In embodiments, the print material comprises a salt buffer. The salt buffer may be phosphate-buffered saline, such as 0.01-100×, preferably 0.1 10×, more preferably 0.5-2× phosphate-buffered saline. The print material preferably has a pH of 5-9, more preferably a pH of 6-8.

### Permanent Support Material

The support material may comprise one or more components that can enhance its structural or biological performance, in particular when the support material is used as permanent support material (i.e. it not separated from the embedded print material after printing).

In an embodiment, the support material comprises one or more crosslinking initiators and/or one or more crosslinking blockers as disclosed herein. The presence of crosslinking agents in the support material allows it to be crosslinked, preferably covalently crosslinked, after embedding the print material. The crosslinking initiator is preferably provided in the support material in an amount of 0.001-5 wt. %, preferably 0.01-1 wt. %, more preferably 0.1-0.5 wt. %, calculated on the total weight of the support material. The support material may comprise one or more salts, wherein the salt is preferably a chloride salt and/or a phosphate salt. In embodiments, the support material comprises a salt buffer. The salt buffer may be phosphate-buffered saline, such as 0.01-100×, preferably 0.1-10×, more preferably 0.5-2× phosphate-buffered saline. The support material preferably has a pH of 5-9, more preferably a pH of 6-8. In an embodiment, the support material comprises one or more biological factors as disclosed herein, preferably one or more biological factors selected from the group consisting of cells, organoids, extracellular vesicles, serum, a serum protein, a growth factor, an extracellular matrix component and decellularized extracellular matrix. In an embodiment, the support material comprises a natural polymer, a synthetic polymer, or a combination thereof, as disclosed herein.

In an embodiment, the print material is removed from the support material (e.g. by flushing), to create channels in the support bath material.

### Method for Preparing the Gelatin Support Material

As part of the invention, a method is provided for preparing a support material for embedded printing. The method preferably comprises dissolving the gelatin as disclosed herein in an aqueous medium to obtain an aqueous gelatin formulation. The aqueous gelatin formulation is preferably in a fluid bulk form and/or a single-phase form. The aqueous gelatin formulation preferably comprises gelatin at a concentration of 0.1-40 wt. %, preferably 1-20 wt. %, more preferably 2-10 wt. %, most preferably 3-7 wt. %, calculated on the total weight of the aqueous gelatin formulation. The gelatin is preferably dissolved in an aqueous buffer, more preferably a salt buffer. In a preferred embodiment, the salt buffer is phosphate-buffered saline, such as 0.01-100×, preferably 0.1-10×, more preferably 0.5-2× phosphate-buffered saline. The buffer preferably has a pH of 5-9, more preferably a pH of 6-8. In an embodiment, the gelatin solution is allowed to gelate. In a preferred embodiment, the gelatin in the formulation is subjected to non-covalent crosslinking. In a preferred embodiment, the gelatin in the formulation is subjected to reversible crosslinking, preferably thermoreversible crosslinking. In a preferred embodiment, the gelatin in the formulation is subjected to physical crosslinking. The gelation and/or crosslinking may be achieved by subjecting the gelatin in the formulation to a temperature of 12-30 °C, preferably 15-25 °C, preferably during 6-18 hours, more preferably 10-14 hours.

In a preferred embodiment, the method for preparing the support material for embedded printing comprises dissolving gelatin as disclosed herein to a concentration of 1-20 wt. % in an aqueous medium to obtain an aqueous gelatin formulation, wherein the gelatin concentration is calculated on the total weight of the aqueous medium, and subjecting the gelatin in the formulation to crosslinking, preferably non-covalent crosslinking, wherein the gelatin in the formulation has an average molecular weight of 50-250 kDa.

As part of the invention, a system is provided suitable for embedded printing as described herein. The system comprises an extruder and the support material disclosed herein. The system may optionally comprise the print material or component(s) thereof as disclosed herein. For instance, the system may further include one or more components of the support material and/or print material disclosed herein selected from the group consisting of the polymer, the crosslinking initiator, the crosslinking blocker, the salt buffer and the biological factor (e.g. cells, an organoid, an extracellular vesicle, serum, a serum protein, a growth factor, an extracellular matrix component and/or decellularized extracellular matrix).

### Gelatin Support Material

In an embodiment, the current invention pertains to a support material for embedded printing obtainable by the method comprising the steps:
a. dissolving gelatin to a concentration of 1-20 wt %, preferably 2-10 wt. %, in an aqueous medium to obtain an aqueous gelatin formulation, wherein the gelatin concentration is calculated on the total weight of the aqueous medium; and
b. subjecting the gelatin in the formulation to non-covalent crosslinking,
wherein the gelatin in the formulation has an average molecular weight of 50-250 kDa, preferably 70-160 kDa, more preferably 80-120 kDa,
wherein the non-covalent crosslinking is preferably by subjecting the gelatin in the formulation to a temperature of 12-30 °C for 6-18 hours.

In an embodiment, the current invention pertains to a support material for embedded printing, wherein the support material is an aqueous gelatin formulation. In an embodiment, the aqueous gelatin formulation comprises 1-20 wt. % gelatin calculated on the total weight of the aqueous gelatin formulation. In an embodiment, the gelatin in the aqueous gelatin formulation has an average molecular weight of 50-250 kDa. In an embodiment, the aqueous gelatin formulation is in a fluid bulk form. In an embodiment, the aqueous gelatin formulation is in a single-phase form.

In an embodiment, the gelatin in the formulation is non-covalently crosslinked. In an embodiment, the aqueous gelatin formulation comprises 2-10 wt. % gelatin calculated on the total weight of the aqueous gelatin formulation. In an embodiment, the gelatin has an average molecular weight of 80-120 kDa.

### General Definitions

The terms ‘comprising’ or ‘to comprise’ and their conjugations are used in the context of the current invention in their non-limiting sense to indicate that items following the word are included, but items not specifically mentioned are not excluded.
Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one’.
In the context of the current invention, a level is considered “increased” or “decreased” when it is at least 1% (such as at least 5%, 10%, 15%, 20%, 25%, 30%, 35%, 40%, 45%, or 50%) higher or lower, respectively, than the corresponding level in a control or reference. In addition or alternatively, a level is considered increased or decreased when it is statistically significantly higher or lower, respectively, compared to a level in a control or reference (including compared to an earlier time point), irrespective of the size of the change. The term “to reduce” may in the context of the current invention be used interchangeably with the term “to decrease”.

### FIGURE LEGENDS

FIG. 1. Comparison of support baths for embedded printing. The top row of pictures show GelMA “90p60” (90 kDa, 60% DOF) being used as a supporting volume in embedded additive manufacturing, as the needle passes through the 90 kDa physical gel no gel-rupture is seen. The bottom row of pictures show GelMA “160p60” (160 kDa, 60% DOF) gelatin being used as a supporting volume in embedded additive manufacturing, as the extrusion needle passes through the 160 kDa physical gel extensive gel-rupture is seen.

FIG. 2. Comparison with support baths prepared from commercially available gelatins. Self-healing behaviour of GelMA support baths prepared from G1890 and G9391 gelatin (Sigma-Aldrich, molecular weight ~160 kDa). It was found that the supporting volumes (gel) are deformed, ripped, and broken, which would not result in a well-defined, or a well-formed, 3D print.

FIG. 3. Comparison of support baths for embedded printing. The four rows show pictures pictures of different gelatins being used as a supporting volume in embedded additive manufacturing. As the needle passes through the ~90 kDa gelatin or GelMA (top two rows, respectively) no gel-rupture is seen. As the needle passes through the ~160 kDa gelatin or GelMA (bottom two rows, respectively) extensive gel-rupture is seen.

FIG. 4. Young's modulus E (A) and Elasticity (B) of GelMAs (5% w/v, 0.1% w/v LAP) with molecular weight (MW) in the range of ~160 kDa (“H”) or ~90 kDa (“L”) and degrees of functionalization (DOF) of 40, 60 or 80%.

FIG. 5. Formation of capillaries after 12 days within ~90 kDa (“Low MW”) or ~160 kDa (“High MW”) GelMA hydrogels seeded with 1.1E7 pancreatic cells. FIG. 4A: DAPI/Phalloidin staining of seeded pancreatic cells. FIG. 4B: quantification of capillary diameter in the edge and the core of the hydrogel.

### EXAMPLES

### Example 1

Example 1 compares the self-healing behaviour of gelatins having different molecular weight.

The gelatin support materials are prepared by dissolving the gelatin to a concentration of 5% (w/v) in 1×PBS and leaving 8-12 to physically crosslink in the dark at room temperature.

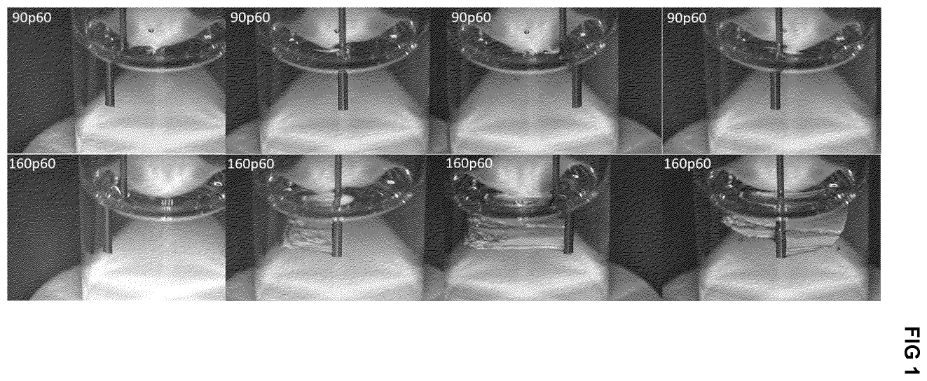

FIG. 1 shows a comparison made in self-healing behaviour of GelMA (60% DOF) with a molecular weight of either ~90 kDa (80-120 kDa range, “90p60”) or ~160 kDa (150-190 kDa range, “160p60”). The suitability of “90p60” or “160p60” support materials for embedded printing is studied by moving a needle in the embedded printing set-up through the support material and studying the amount of deformation or self-healing capacity of the support material.

FIG. 1 shows that the molecular weight of the gelatin influences the self-healing capacity of the supporting material. The top four figures in Fout! Verwijzingsbron niet gevonden. show a needle that moves through the “90p60” GelMA supporting volume. The bottom four pictures show the same needle moving through a “160p60” GelMA supporting volume. The smear that appears in the bottom row pictures indicates that the supporting volume (gel) is deformed, ripped, and broken, which would not result in a well-defined, or a well-formed, 3D print. The “90p60” GelMA shows no signs of gel-rupture, leading to high-definition 3D prints.

Preliminary experiments indicated that gelatins with an average molecular weight of 120 kDa or 140 kDa appear to have desirable self-healing behaviour similar to the “90p60” gelatin.

It was considered that a loss in self-healing behaviour may occur if a certain molecular weight threshold is exceeded. To include gelatins with relatively higher molecular weights, further comparisons were made with commercially available gelatins G1890 and G9391 (both from Sigma-Aldrich). Since it was found that the true average molecular weight of commercially-available gelatins may deviate from the (expected) average molecular indicated by the supplier, the molecular weight of the gelatins were measured prior to the experiments.

G1890 (Sigma-Aldrich) is a type A gelatin from porcine skin. The average molecular weight was measured to be ~160 kDa, which is in agreement with measurements in earlier reports (Olijve et al. Stem cell proliferation on 3D printed discs improved with low endotoxin and low molecular weight gelatin coating. Rousselot B V. October 2021)

G9391 (Sigma-Aldrich) is a type B gelatin from bovine skin. The average molecular weight was measured to be ~160 kDa.

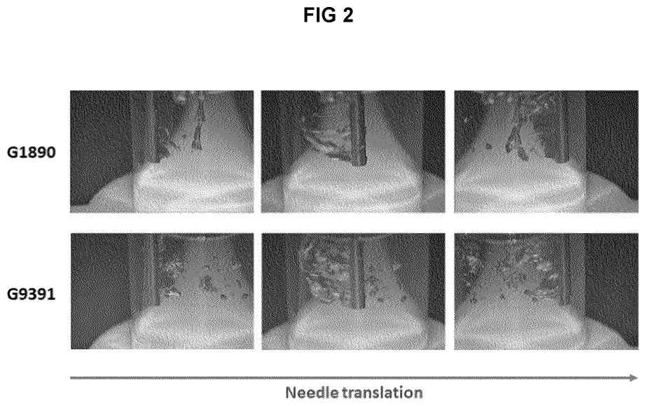

FIG. 2 shows the self-healing behaviour of G1890 and G9391. It was found that the supporting volume (gel) is deformed, ripped, and broken, which would not result in a well-defined, or a well-formed, 3D print.

FIG. 2 further shows that the molecular weight of the gelatin influences the self-healing capacity of the supporting material and that an average molecular weight of 160 kDa or more is undesirable.

### Example 2

Example 2 compares the self-healing behaviour of four different gelatins:
1) non-modified gelatin with a molecular weight of ~90 kDa (18-120 kDa range, “90 kDa”);
2) GelMA with a molecular weight of ~90 kDa and 60% DOF (“90p60”);
3) non-modified gelatin with a molecular weight of ~160 kDa (150-190 kDa range, “160 kDa”);
4) GelMA with a molecular weight of ~160 kDa and 60% DOF (“160p60”).

The gelatin support material is prepared by dissolving the gelatins in to a concentration of 5% (w/v) in 1×PBS) and leaving overnight to gel in the dark at room temperature.

The suitability of the four support materials is studied by moving a needle in the embedded printing set-up through the support material and studying the amount of deformation or self-healing capacity of the support material.

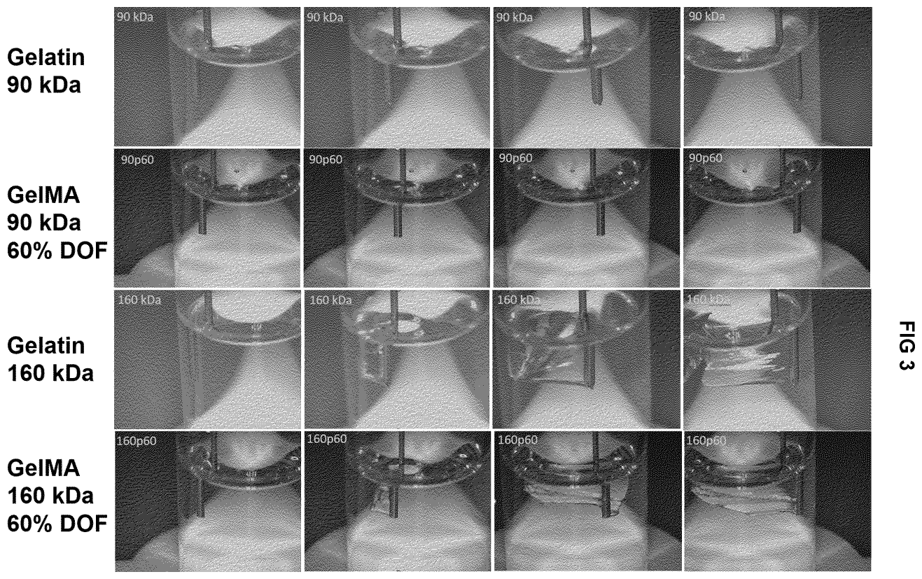

FIG. 3 shows that the molecular weight of the gelatin influences the self-healing capacity of the supporting material. The non-modified gelatin or GelMA with a molecular weight of ~90 kDa, show a needle that moves through the supporting volume without signs of gel-rupture, leading to high-definition 3D prints.

The non-modified gelatin or GelMA with a molecular weight of ~160 kDa show a smear that indicates that the supporting volume (gel) is deformed, ripped, and broken, which would not result in a well-defined, or a well-formed, 3D print.

Similar results are seen for modified gelatins other than GelMA, including gelatin desaminotyrosine (GelDAT).

The results show that the self-healing capacity of gelatin support materials is dependent on the molecular weight. Furthermore, the results show that both non-modified gelatins and different modified gelatins have self-healing capacity in support materials when appropriate molecular weight is selected.

### Example 4

Example 4 compares different support materials in embedded printing.

### Method

A carbopol support material is prepared based on the protocol described by Bhattacharjee et al. (Sci Adv. 2015 Sep. 25; 1(8):e1500655). In short, a powdered Carbopol (0.7%, w/v) is dispersed into cell growth media at 37 °C under sterile conditions, and the gel medium is incubated at 37 °C and 5% CO₂ for 24 hours.

A covalently crosslinked, thermo-irreversible, gelatin support material is prepared based on the protocol described by Pitton et al. (Materials Letters. Volume 341, 15 Jun. 2023). In short, GelMA (~90 kDa, 60% DOF) is dissolved in DPBS (Dulbecco's Phosphate Buffered Saline) (10 wt. %) together with Irgacure 2959 (0.05% v/v), and subsequently crosslinked by UV light (λ=405 nm, 120 s).

A gelatin particle/colloidal support material (“gelatin slurry”) is prepared based on the protocol described by Hinton et al. (Sci Adv. 2015 Oct. 23; 1(9):e1500758). In short, gelatin (4.5% w/v) is mixed with CaCl₂ and allowed to gel for 12 hours at 4 °C. CaCl₂ is added at 4 °C and the contents is blended. The soluble gelatin is removed by centrifugating and the gelatin particulate/colloidal bath (slurry) is obtained.

A physically crosslinked, fluid bulk, gelatin support material is prepared according to Example 1 using non-functionalized gelatin or using GelMA (~90 kDa, 60% DOF).

### Result

Table 1 shows the comparison of different support materials in embedded printing.

The physically crosslinked gelatin support material shows good yield-stress behavior and self-healing capacity. Compared to the other support materials, the physically crosslinked gelatin support material can be obtained more practically and conveniently, by dissolving the gelatin in a salt buffer and allowing crosslinking under physiological conditions.

The physically crosslinked, fluid bulk, gelatin support materials is more advantageous than the other support materials tested in terms of ease of production (not requiring special knowledge or complex protocol), a short preparation time, high reproducibility, and high scalability of the method (e.g. from lab to clinical setting).

The use of physically crosslinked gelatin does not rely on functionalised gelatins, which reduces the costs and allows larger volumes of support materials to be used.

In comparison to covalently crosslinked gelatin, the (thermos)reversibility of the physical crosslinking of the gelatin is advantageous, because the support material can be easily separated from the print material (for instance by simple melting).

Gelatin furthermore is found to be biocompatible and avoids the toxicity that can be seen with chemicals such as Carbopol.

TABLE 1
Comparison of different support materials in embedded printing.

| | | | | Ease of | | |
|---|---|---|---|---|---|---|
| | Preparation | Repro- | Self-healing | extraction | | Volume of |
| Support material | time | ducibility | capacity | of print | Scalability | print bath |
| Carbopol | Average | High | Average | Average | Low | Low |
| Covalenty | Average | Average | Average | Average | Low | Low |
| crosslinked GelMA | | | | | | |
| Gelatin | High | Average | Average | Low | Low | Low |
| particles/colloidal | | | | | | |
| Physically | Short | High | High | High | High | High |
| crosslinked, | | | | | | |
| fluid bulk gelatin | | | | | | |
| Physically | Short | High | High | High | High | High |
| crosslinked, | | | | | | |
| fluid bulk GelMA | | | | | | |

### Example 5

Example 5 shows the effect of molecular weight on mechanical and vasculogenic properties of GelMA hydrogels.

### Methods

### Preparation of Hydrogel Precursors

Hydrogel precursors are prepared based on GelMA, phosphate-buffered saline (PBS), and Lithium phenyl (2,4,6-trimethylbenzoyl) phosphinate (LAP).

A first method involves sterile filtration. A second method does not involve sterile filtration.

Method 1—with Sterile Filtration:
1. Dissolve GelMA in 0.1×PBS (up to 2.5% w/v GelMA)
a. Weigh 10 g of GelMA
b. Add 400 mL of 0.1×PBS
c. Place for 1 h in the oven at 45 °C
d. Swirl and vortex
e. Place for 1 h in the oven at 45 °C
2. Sterile filter the solution over a 0.45 μm filter
3. Place sterile solution in the oven (e.g. at 45 °C) for ~30 minutes
4. Sterile filter the solution over a 0.22 μm filter—in the laminar flow cabinet
5. Make 50 mL aliquots, using falcon tubes with filter caps
6. Let the aliquots cool down to room temperature
7. Place aliquots in the freezer—freezer over the course of several days
8. Place aliquots in the Freeze dryer (pressure: 0.37 mbar)—the filter caps allow gas exchange (water vapor), while keeping the GelMA sterile—freeze dry for several days

The GelMA is sterilized and dried ready to be redissolved (e.g. in filter sterilized 0.9×PBS-LAP stock solution, e.g. 1 mg/mL LAP) at desired concentration

### Method 2—Without Sterile Filtration:

Dissolve GelMA in 1.0×PBS-LAP stock solution.

Hydrogels [5% (w/v) GelMA, 0.1% LAP (w/v), 5×10⁶ 11E7 cells/ml precursor] are prepared using GelMA (60% DOF) with a molecular weight of either ~90 kDa (80-120 kDa range, “90p60”) or ~160 kDa (150-190 kDa range, “160p60”). The hydrogels are subjected to the a mechanical test and a vascularization assay. Pancreas tissue is used as reference material.

### Vascularization Assay

### Materials Used:

Alamar Blue staining kit;
Calcein-AM and Propidium Iodide (PI) staining kit;
4′,6-diamidino-2-phenylindole (DAPI)
Alexa Fluor 488 phalloidin probe
Fluorescence—absorbance plate reader (Tecan AG);
Cells: 1.1E7 cell line, (induced) human mesenchymal stem cells (MSC), human umbilical vein endothelial cells (HUVEC).

### Cell Culturing:

Cells are seeded into GelMA hydrogels at desired concentration (~1×10⁶-1×10⁷ cells/ml).

GelMA hydrogels are prepared according to Examples 1-2.

The hydrogels are cultured in cell culture medium at 37 °C/5.0% CO₂.

### Read-outs:

Alamar Blue staining and fluorescence staining is performed on various days (e.g. period day 1-14) and measured using a fluorescence plate-reader according to the manufacturer's instructions. This allows determination of indirect and direct cytotoxicity and cell proliferation. Vascularization is scored on various days (e.g. period day 1-14) according to the vascular network formation, total length of vessel-like structures, presence of intercellular junctions, endothelial cell-related phenotype (CD31 staining, VE-cadherin, alpha smooth muscle actin). Cell adherence testing is performed on various days (e.g. day 1-14).

### Elasticity Testing

For elasticity testing, the samples were washed in PBS at 37 °C overnight to reach equilibrium swelling. To assess the viscoelastic properties, a strain recovery measurement was performed at a constant 20% strain for 2 minutes and then left for recovery for 1 minute, with a preload force of 0.0010 N. The elasticity index was calculated as the ratio between the recovered stress and the maximal stress under constant strain (value after 2 mins/highest value*100).

### Results

Hydrogels prepared with GelMA with “90p60” lead to best mechanical properties resembling native soft tissue (lower stiffness, higher stress relaxation) and best vasculogenic response. This further shows that an appropriate average molecular weight is advantageous for the hydrogel properties.

TABLE 2
hydrogels are prepared with GelMA starting materials with ~90
kDa or ~160 kDa molecular weight ranges and a DOF of ~60% (precursor:
5% (w/v) GelMA, 0.1% (w/v) LAP, 5 × 10⁶ 11E7 cells/ml).

| | Soft tissue | |
|---|---|---|
| Molecular weight | resemblance | Vascularization |
| ~90 kDa (range | High | High |
| 80-120 kDa) | | |
| ~160 kDa (range | Low | Average/Low |
| 150-190 kDa) | | |

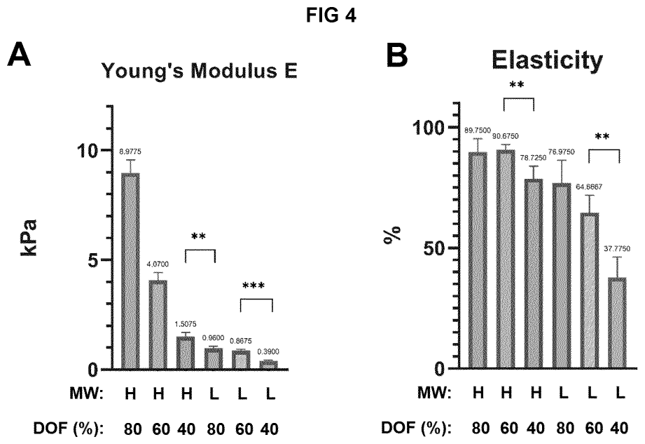

FIG. 4 shows the mechanical characterization (Young's modulus E and elasticity) of GelMAs (5% w/v, 0.1% w.v LAP) with molecular weight (MW) in the range of ~160 kDa (“H”) or ~90 kDa (“L”) and degrees of functionalization (DOF) of 40, 60 or 80%. It is found that compressive mechanical properties increase with the MW and the DOF %. Elasticity is generally high (e.g. more than 70%) for all the samples except for MW of ~90 kDa in combination with 60% DOF or 40% DOF.

Pancreas tissue was used as reference material in the mechanical tests, being a typical target tissue to mimic. The defrosted pancreas had an average elasticity of 16.8±5.5% The fresh pancreases had an average elasticity of 35.5±4.7% and 30.7±8.4%.

The MW of ~90 kDa, particularly in combination with ~40-60% DOF was able to achieve a hydrogel recapitulating a soft tissue as the pancreatic one.

These results suggest that the intermolecular interactions are affected by the total average length of the GelMA chains, hence the ~90 kDa GelMAs show a different trend in hydrogel strength in relation to GelMA concentration in the precursor phase than the ~160 kDa GelMAs.

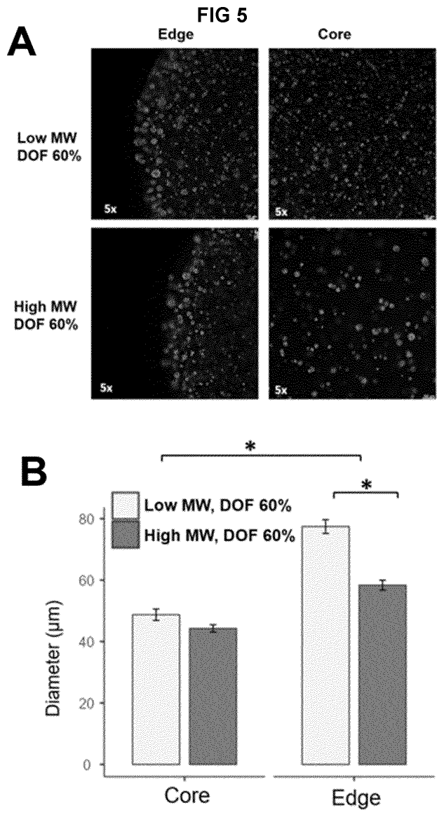

FIG. 5 shows the formation of capillaries of different dimensions within the hydrogel starting to form at day 12 of culture following seeding with 1.1E7 pancreatic cells. The GelMA with MW of ~90 kDa showed larger and more mature capillaries than the GelMA with MW of ~160 kDa. The formation of capillaries was highest for the DOF in the range of 40-60%. These results further confirm that ~90 kDa GelMA hydrogels, in particular with DOF in the range of 40-60% might recapitulate an environment surprisingly leading to self-vascularization.

1. A method for embedding a print material in a support material, the method comprising the step of depositing the print material into the support material,
wherein the support material is an aqueous gelatin formulation comprising gelatin in an amount of 1-10 wt. %, calculated on the total weight of the support material,
wherein the gelatin in the aqueous gelatin formulation has an average molecular weight of 50-140 kDa.

2. The method according to claim 1, wherein the gelatin has an average molecular weight of 60-140 kDa.

3. The method according to claim 2, wherein the gelatin has an average molecular weight of 70-130 kDa.

4. The method according to am claim 1, wherein the aqueous gelatin formulation is in a fluid bulk form and/or in a single-phase form.

5. The method according to claim 1, wherein the gelatin in the formulation is non-covalently crosslinked.

6. The method according to claim 1, wherein the gelatin is chemically functionalized gelatin selected from the group consisting of gelatin methacryloyl, gelatin desaminotyrosine, gelatin methacrylamide, gelatin acryloyl, gelatin aminoethyl, gelatin-methacryloyl-aminoethyl, gelatin styrene, gelatin tyramine, gelatin-methacrylamidedopamine, gelatinmethacryloyl-acyl, gelatin-acrylamide, gelatin-3,30,4,40-benzophenone tetra carboxylic dihydroxyethylmethacrylate, gelatin with boc-protected primary amines and aminoethylmethacrylate coupled to the carboxylic acids, methacrylated poly(ethylene glycol)-modified gelatin, aminated gelatin, aminated-thiolated-gelatin, gelatin modified with cysteine and 2-mercaptopyrimidine-4,6 diol, gelatin-cys, gelatin-poly(ethylene glycol) cysteine, gelatinthiobutylamidine modified with 2-mercaptonicotinic acid, thiolated gelatin, gelatin-thiobutyrolacton, gelatin-tyramine, gelatin/tyramine/heparin, gelatin furfuryl amine, gelatin furfuryl isocyanate, gelatin furfuryl glycidyl ether, gelatin-5-[2-(5-methyl furylene vinylene)]furancarboxyaldehyde, gelatin-anthracene, gelatin-nitrocinnamate, gel-furan, gelatin furfuryl glycidyl ether, gelatin-norbornene, gelatin tetrazine, gelatin-pentenoate, gelatin allylglycidyl ether, gelatin-vinylester, gelatin methacryloyl (GelMA) and/or gelatin desaminotyrosine (GelDAT) and combinations thereof.

7. The method according to claim 1, wherein the support material comprises 3-7 wt. % gelatin, calculated on the total weight of the support material.

8. The method according to claim 1, wherein the print material comprises a hydrogel precursor, wherein the hydrogel precursor preferably comprises a natural polymer, a synthetic polymer, or any combination thereof.

9. The method according to claim 1, wherein the support material comprises one or more crosslinking initiators and/or one or more crosslinking blockers.

10. The method according to claim 1, wherein the support material and/or the print material comprises one or more biological factors selected from the group consisting of cells, organoids, extracellular vesicles, serum, a serum protein, a growth factor, an extracellular matrix component and decellularized extracellular matrix.

11. The method according to claim 1, wherein the print material is embedded in the support material at a location where the printed material is deposited.

12. Use of gelatin in a support material for embedded printing,
wherein the support material is an aqueous gelatin formulation comprising gelatin in an amount of 1-10 wt. %,
wherein the gelatin has an average molecular weight of 50-140 kDa.

13. Use according to claim 12, wherein the gelatin has an average molecular weight of 60-140 kDa.

14. Use according to claim 13, wherein the gelatin has an average molecular weight of 70-130 kDa.

15. Use according to claim 12, wherein the support material is in a fluid bulk form and/or in a single-phase form.

16. Use according to claim 12, wherein the gelatin is non-covalently crosslinked.

17. Use according to claim 12, wherein the gelatin is chemically functionalized gelatin, preferably selected from the group consisting of gelatin methacryloyl, gelatin desaminotyrosine, gelatin methacrylamide, gelatin acryloyl, gelatin aminoethyl, gelatin-methacryloyl-aminoethyl, gelatin styrene, gelatin tyramine, gelatin-methacrylamidedopamine, gelatinmethacryloyl-acyl, gelatin-acrylamide, gelatin-3,30,4,40-benzophenone tetra carboxylic dihydroxyethylmethacrylate, gelatin with boc-protected primary amines and aminoethylmethacrylate coupled to the carboxylic acids, methacrylated poly(ethylene glycol)-modified gelatin, aminated gelatin, aminated-thiolated-gelatin, gelatin modified with cysteine and 2-mercaptopyrimidine-4,6 diol, gelatin-cys, gelatin-poly(ethylene glycol) cysteine, gelatinthiobutylamidine modified with 2-mercaptonicotinic acid, thiolated gelatin, gelatin-thiobutyrolacton, gelatin-tyramine, gelatin/tyramine/heparin, gelatin furfuryl amine, gelatin furfuryl isocyanate, gelatin furfuryl glycidyl ether, gelatin-5-[2-(5-methyl furylene vinylene)]furancarboxyaldehyde, gelatin-anthracene, gelatin-nitrocinnamate, gel-furan, gelatin furfuryl glycidyl ether, gelatin-norbornene, gelatin tetrazine, gelatin-pentenoate, gelatin allylglycidyl ether, gelatin-vinylester, gelatin methacryloyl (GelMA) and/or gelatin desaminotyrosine (GelDAT) and combinations thereof.

18. Use according to claim 12, wherein the support material is a temporary support material or a permanent support material.

19. Use according to claim 12, wherein the embedded printing is extrusion-based embedded printing and/or in bioprinting.

20-31. (canceled)
